(19) United States
(12) Patent Application Publication
(10) Pub. No.: US 2004/0033182 A1
(43) Pub. Date: Feb. 19, 2004

(54) Oral hygiene device sanitizer

(76) Inventor: Syntheia L. Duffy, Beaumont, TX (US)

Correspondence Address: William P. Glenn, Jr., Suite 205, 2102 Mechanic Street, Galveston, TX, 77550, US

(21) Appl. No.: 10/638,451

(22) Filed: Aug. 11, 2003

Related U.S. Application Data
(60) Provisional application No. 60/403,406, filed on Aug. 14, 2002.

(51) Int. Cl. A61L 2/07

(52) U.S. Cl. 422/307; 422/26

(57) ABSTRACT

An oral hygiene device sanitizer having a flue with a heating region distal to a sterilization region, wherein an oral hygiene device chamber is located in a sterilization region. A controllable heater fixed in a flue heats up air to no more than 220 degree Fahrenheit which flows across an oral hygiene device (such as a toothbrush, gum stimulator or tongue scraper) for at least forty-five minutes. In another embodiment, a water chamber is positioned adjacent to a controllable heater to generate steam to be mixed with heated air and further enhance the sanitization of an oral hygiene device.

### CROSS REFERENCE

This application further claims the benefit of Provisional Application No. 60/403,406 filed Aug. 14, 2002. This cross reference is not admitted to be prior art with respect to the present invention by its mention in the cross reference and background sections. Furthermore, the entire disclosures of the previous application are to be considered a part of this disclosure and is hereby incorporated by reference.

### DESCRIPTION

1. Technical Field

The present invention relates generally to the sanitization of oral hygiene devices used by consumers. More specifically the present invention relates to a sanitizer which uses dry heat or steam to sanitize a toothbrush, gum stimulator or tongue scrapper.

2. Background Art

In general, toothbrushes and other oral hygiene devices are designed and manufactured to assist and enable a consumer practice proper oral hygiene. Unfortunately, the modern practice of oral hygiene does not focus or emphasize proper storage, cleaning or sanitation of these oral hygiene devices. Such oversight encourages a toothbrush or other oral hygiene devices to be left unclean or stored in ways which may introduce infectious materials into the mouth that may lead to gum disease, heart disease and/or an increase in blood-glucose levels, a potentially life threatening situation to diabetics.

Existing practice typically calls for a person's toothbrush (or other oral hygiene device) to be stored in a holder (e.g. cup or toothbrush holder) after placing it under a stream of running water to loosen and flush away debris (and tooth paste). Failure to do so increases the risk of re-introducing that which a person is trying to remove (be it food, plaque or organisms which produce plaque and/or bad breath). But even the existing practice of cleaning with running water, does not significantly eliminate the organisms which produce plaque, bad breath and/or other diseases.

In view of the above described deficiencies associated with the cleaning of oral hygiene devices, the present invention has been developed to alleviate these drawbacks and provide further benefits to the user. These enhancements and benefits are described in greater detail hereinbelow with respect to several alternative embodiments of the present invention.

### SUMMARY OF THE INVENTION

The present invention in its several disclosed embodiments alleviates the drawbacks described above with respect to conventional cleaning of oral hygiene devices and incorporates several additionally beneficial features.

It is one object of the present invention to provide a device which cleans oral hygiene devices like toothbrushes, gum stimulators and tongue scrappers.

Its is another object of the present invention to provide a device which sanitizes oral hygiene devices like toothbrushes, gum stimulators and tongue scrappers.

It is a further object of the present invention to provide a device which sterilizes oral hygiene devices like toothbrushes, gum stimulators and tongue scrappers.

The beneficial effects described above apply generally to each of the exemplary devices and mechanisms disclosed herein of the oral hygiene device sanitizer. The specific structures through which these benefits are delivered will be described in detail hereinbelow.

### BRIEF DESCRIPTION OF THE DRAWINGS

The invention will now be described in greater detail in the following way of example only and with reference to the attached drawings, in which:

FIG. 1 is a cross sectional view of an oral hygiene device sanitizer.

FIG. 1a is a cross sectional view of another embodiment of an oral hygiene device sanitizer.

FIG. 2 is a perspective view of another embodiment of an oral hygiene device sanitizer.

FIG. 3 is a cross sectional view of an oral hygiene device sanitizer with a water vessel.

FIG. 4 is a cross sectional view of an oral hygiene device sanitizer with another embodiment of a water vessel.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale, some features may be exaggerated or minimized to show details of particular components. Therefore, specific structural and functional details disclosed herein are not to be interpreted as limiting, but merely as a basis for the claims and as a representative basis for teaching one skilled in the art to variously employ the present invention.

Furthermore, elements may be recited as being “coupled”; this terminology's use contemplates elements being connected together in such a way that there may be other components interstitially located between the specified elements, and that the elements so specified may be connected in fixed or movable relation one to the other. Certain components may be described as being “adjacent” to one another. In these instances, it is expected that a relationship so characterized shall be interpreted to mean that the components are located proximate to one another, but not necessarily in contact with each other. Normally there will be an absence of other components positioned therebetween, but this is not a requirement. Still further, some structural relationships or orientations may be designated with the word “substantially.” In those cases, it is meant that the relationship or orientation is as described, with allowances for variations that do not effect the cooperation of the so described component or components.

Referring to FIG. 1, an oral hygiene device sanitizer 10 in its most basic form includes a flue 20 with a heating region 30 distal to a sterilization region 50. Within a sterilization region 50 is an oral hygiene device chamber 52. A controllable heater 32 is fixed in a flue 20 above an inlet 31 at a heating region 30 to heat up air within a flue 20 to no more than approximately 220 degrees Fahrenheit. An oral hygiene device holder 54 is fixed to an oral hygiene device chamber 52 to removably fix at least a portion of an oral hygiene device within said heated air for at least forty-five (45) minutes for sanitization. A removably fixed lid 22 is attached to a flue 20 to provide access to an oral hygiene device holder 54. A restrictor 26 located at an end of a sterilization region 50 reduces the flow of heated air out of a sterilization region 50. A base mount 60 is fixed to a flue 20 to position and stabilize an oral hygiene device to a work surface 03, such as a counter top (FIG. 1) or wall (FIG. 1a).

A flue 20 is constructed or formed from a material or materials which can retain a given shape at temperatures of at least about 220 degrees Fahrenheit, such as stainless steel or plastic. It is contemplated that such a heat resistant material has a non-porous surface to prevent or at least eliminate the collection of surface water and/or debris within a sterilization region 50. Such a surface further promotes ease of cleaning. In a preferred embodiment, a flue 20 has a cylindrical shape with an inner diameter and length sufficient to contain a controllable heater 32, and an oral hygiene device 05.

In a preferred embodiment, insulation 28 is disposed between inner and outer walls of a flue 20 to prevent transfer of heat from said heated air to an exterior surface of a flue 20 (See FIGS. 1 and 1a). Insulation 28 can be in the form of an insulative material such a rock wool, spun fiberglass, styrofoam or a vacuum.

A base mount 60 is constructed or formed from a material or materials capable of securing an oral hygiene device sanitizer 10 to an adjacent work surface and aligning a flue 20 in a generally vertical position. Securement of an oral hygiene device sanitizer 10 can be as simple as a broad weighted base mount 60 removably fixed to a horizontal work surface (like a counter) or a base mount 60 capable of being securingly attached to a work surface (like a wall)) with fasteners (like screws, nails, adhesives and/or suction cups). It is further contemplated that a base mount 60 be pivotally attached to a flue 20 to allow a user to position a flue 20 between a range of vertical to about thirty (30) degrees off horizontal (not shown).

Referring to FIG. 1, a controllable heater 32 is fixed in a flue 20 at a heating region 30 to heat up air within a flue 20 to no more than about 220 degrees Fahrenheit.

In a preferred embodiment, controllable heater 32 is electric (AC or DC) and is controlled by a microprocessor (not shown) with a ground fault interruption circuit (not shown).

Likewise, controllable heater 32 is contemplated to have a number or preset heating temperatures (e.g. high, medium and low) which can be controlled by a microprocessor or a user. The shape of a controllable heater 32 is dictated by the shape of the inner surface of a flue 20 and the need to maximize the heatable surface area over which air flows. It is further contemplated that heat from a controllable heater 32 is derived from a catalytic heat process using a flammable liquid (not shown).

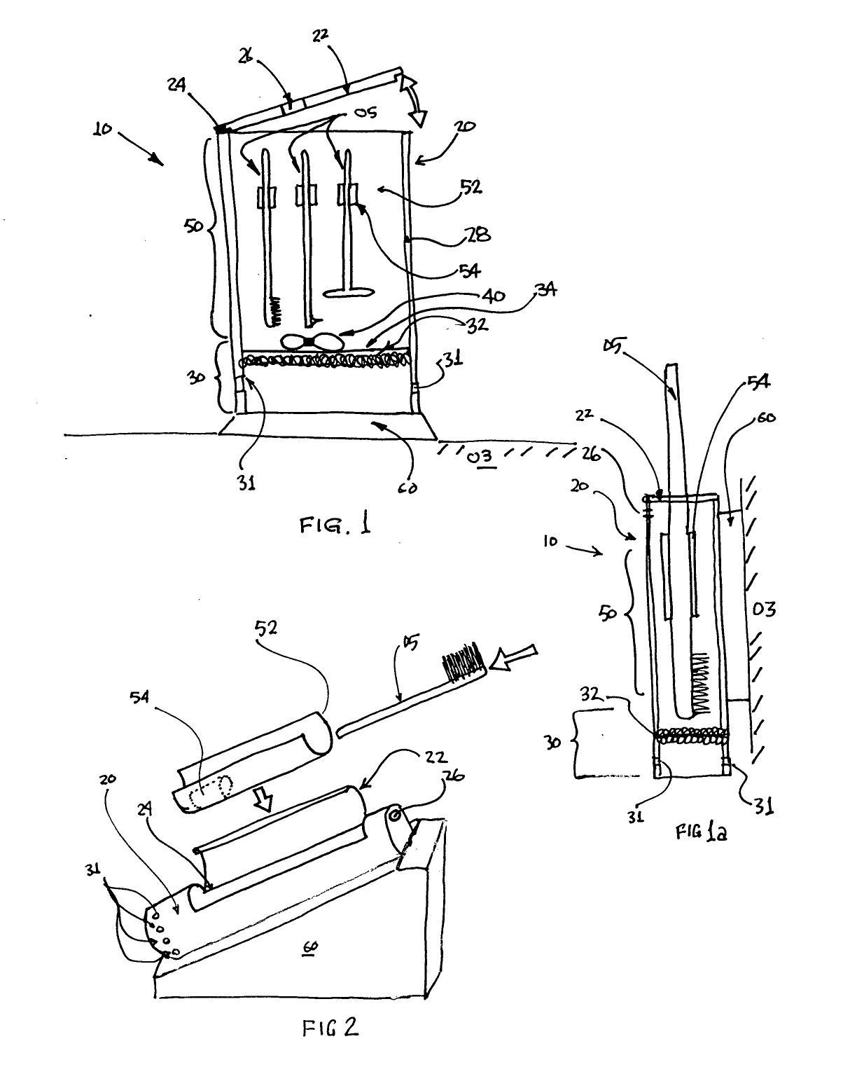

An oral hygiene device chamber 52 (See FIG. 1) is fixed in a flue 20 at a sterilization region 50 to receive and hold an oral hygiene device 05. In a preferred embodiment, oral hygiene device chamber 52 is permanently fixed in a flue 20 and accessible by a removably fixed lid attached to a flue 20. In another preferred embodiment, oral hygiene device chamber 52 is removably fixed in a flue 20 (See FIG. 2). In either embodiment, a holder 54 is fixed to an oral hygiene device chamber 52 to releasably mount at least a part of an oral hygiene device 05 into a flow of heated air (See FIG. 1a). In another embodiment, holder 52 mounts the complete oral hygiene device 05 into a flow of heated air (See FIG. 1). In a preferred embodiment, holder 54 is a clip with arms set a small distance apart which flex to receive, hold at high temperatures and release an oral hygiene device 05 (See FIG. 1). In another embodiment, holder 54 is a cup with an open end configured to receive an oral hygiene device 05 (See FIG. 2). Both holder 54 and chamber 52 are contemplated to be constructed from materials that are able to withstand temperatures of at least 220 degrees Fahrenheit. Finally it is contemplated that an oral hygiene device chamber 52 can constructed to hold up to six oral hygiene devices 05 at one time for sanitization.

As depicted in FIG. 1, a removably fixed lid 22 is fixed to an end of a flue 20. In another embodiment, removably fixed lid 22 is fixed to cover an opening formed in a side wall of a flue 20 (See FIG. 2). A removably fixed lid 22 provides access to a holder 52.

In a preferred embodiment, removably fixed lid 22 is attached to a flue by a hinge 24. A removably fixed lid 22 is contemplated to be constructed from materials that are able to withstand temperatures of at least 220 degrees Fahrenheit.

As depicted in FIG. 1, a restrictor 26 is located at an end of a sterilization region 50 of a flue 20 to reduce heated air from flowing out of a sterilization region 50. A restrictor 26 in its simplest form is cap with a small opening formed in an end region of a sterilization region 50 to significantly reduce the flow of air through a flue 20. The air flow through a restrictor 26 is between about half and one third of a flow through inlet 31.

In a preferred embodiment as shown in FIG. 1, a controllable fan 40 is fixed in a flue 20 above a controllable heater 32 to enhance flow of heated air around at least a portion of an oral hygiene device 05 mounted in a holder 54. It is contemplated that control of fan 40 is carried out by existing control devices such as a thermostat, timer or a combination of both. It is further contemplated that a controllable fan 40 have variable speeds.

In another embodiment as shown in FIG. 1, a heat diffuser 34 is positioned adjacent to a controllable heater 32 to enhance even heating of air flowing through a flue 20. A heat diffuser 34 is constructed of materials which conduct and dissipate heat evenly over its surface without absorbing much heat, like aluminum or steel.

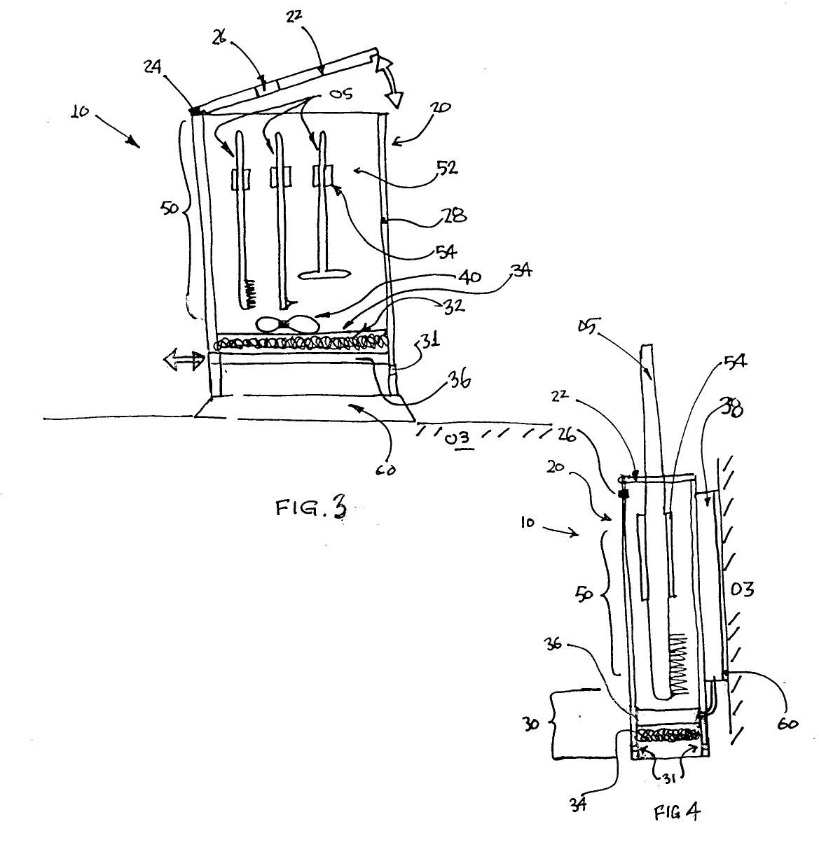

In a preferred embodiment as shown in FIGS. 3 and 4, a water vessel 36 is fixed adjacent to a controllable heater 32 to boil at least two (2) ounces of water to humidify heated air with steam. The steam enhances the sanitation of an oral hygiene device 05. Water vessel 36 can be fixed above (FIG. 4), below (FIG. 3) or next (not shown) to a controllable heater 32. Furthermore, water vessel 36 can be positioned below a controllable heater 32 such that steam flows past the controllable heater 32 (FIG. 3). In another embodiment, water vessel 36 is positioned below a controllable heater 32 such that steam is ducted around the controllable heater 32 by a heat diffuser 34. Water vessel 36 is contemplated to be constructed of the same material as used to construct a heat diffuser 34. As shown in FIG. 3, water vessel 36 is removably fixed and accessible through an opening in a flue 20. As shown in FIG. 4, water vessel 36 is fluidly connected to a water reservoir 38 accessible to a user.

An oral hygiene device sanitizer and its components have been described herein. These and other variations, which will be appreciated by those skilled in the art, are within the intended scope of this invention as claimed below. As previously stated, detailed embodiments of the present invention are disclosed herein; however, it is to be understood that the disclosed embodiments are merely exemplary of the invention that may be embodied in various forms.

What is claimed and desired to be secured by Letters Patent is as follows:

1. A oral hygiene device sanitizer comprising:
a flue with a heating region distal to a sterilization region, said sterilization region having an oral hygiene device chamber;
a controllable heater fixed in said flue above an inlet at said heating region to heat up air within said flue to no more than approximately 220 degrees Fahrenheit;
an oral hygiene device holder fixed to said oral hygiene device chamber to removably fix at least a portion of an oral hygiene device within said heated air for at least forty-five minutes for sanitization;
a base mount fixed to said flue;
a removably fixed lid attached to said flue to provide access to said oral hygiene device holder; and
a restrictor located at an end region of said sterilization region to reduce said heated air from flowing out of said sterilization region.

2. A oral hygiene device sanitizer as claimed in claim 1, further comprising a controllable fan fixed in said flue above said controllable heater to enhance flow of said heated air around said at least a portion of an oral hygiene device.

3. A oral hygiene device sanitizer as claimed in claim 1, further comprising insulation about said flue to prevent transfer of heat from said heated air to an exterior surface of said flue.

4. A oral hygiene device sanitizer as claimed in claim 1, further comprising a heat diffuser positioned adjacent to said controllable heater to enhance even heating of said air.

5. A oral hygiene device sanitizer as claimed in claim 1, further comprising a water vessel fixed adjacent to said controllable heater to boil at least 2 ounces of water to humidify said heated air with steam.

6. A oral hygiene device sanitizer as claimed in claim 5, wherein said water vessel is removably fixed adjacent to said controllable heater and accessible through an opening in said flue.

7. A oral hygiene device sanitizer as claimed in claim 5, wherein said water vessel is fluidly connected to a water reservoir accessible to a user.

8. A oral hygiene device sanitizer as claimed in claim 5, wherein said water vessel is fixed above said controllable heater.

9. A oral hygiene device sanitizer as claimed in claim 5, wherein said water vessel is fixed below said controllable heater and said steam flows past said controllable heater.

10. A oral hygiene device sanitizer as claimed in claim 5, wherein said water vessel is fixed below said controllable heater and said steam is ducted around said controllable heater.
